(19) United States
(12) Patent Application Publication
(10) Pub. No.: US 2019/0000301 A1
(43) Pub. Date: Jan. 3, 2019

(54) CONTROLLER

(71) Applicant: OLYMPUS CORPORATION, Tokyo (JP)

(72) Inventors: Fumiyuki ONODA, Tama-shi (JP); Yasuaki NATORI, Akishima-shi (JP); Keijiro OMOTO, Hachioji-shi (JP); Takashi YAMASHITA, Hachioji-shi (JP); Takashi SUZUKI, Hino-shi (JP); Yoshitaka UMEMOTO, Hachioji-shi (JP)

(73) Assignee: OLYMPUS CORPORATION, Tokyo (JP)

(21) Appl. No.: 16/124,449

(22) Filed: Sep. 7, 2018

Related U.S. Application Data
(63) Continuation of application No. PCT/JP2017/013970, filed on Apr. 3, 2017.

(30) Foreign Application Priority Data
May 26, 2016 (JP) 2016-105207

(51) Int. Cl. A61B 1/00; A61B 1/018; A61B 18/14

(52) U.S. Cl. CPC A61B 1/00006; A61B 1/00018; A61B 1/00156; A61B 2018/00982; A61B 1/018; A61B 18/1492; A61B 1/00055; A61B 1/00016

(57) ABSTRACT

A controller controls a first device, one of an endoscope and a treatment instrument being the first device, a remaining one of the endoscope and the treatment instrument being a second device, the endoscope comprising an insertion tube and a self-propelled mechanism adapted to generate force for insertion or removal of the insertion tube. The controller includes a determination unit configured to determine whether or not the second device is functioning; and a control unit configured to restrict an operation of the first device if the second device is determined to be functioning.

### CROSS-REFERENCE TO RELATED APPLICATIONS

This application is a Continuation Application of PCT Application No. PCT/JP2017/013970, filed Apr. 3, 2017 and based upon and claiming the benefit of priority from prior Japanese Patent Application No. 2016-105207, filed May 26, 2016, the entire contents of all of which are incorporated herein by reference.

### BACKGROUND OF THE INVENTION

### 1. Field of the Invention

The present invention relates to a controller.

### 2. Description of the Related Art

Generally, insertion devices such as an endoscope effectuate the insertion into, for example, a lumen through their elongated insertion tubes. Among such insertion devices intended for the insertion into a lumen, a so-called self-propelled type is known.

For example, International Publication No. 2015/118773 describes an endoscope system that includes a rotating self-propelled type endoscope. This rotating self-propelled type endoscope includes a rotating tube with helical fins, called a power spiral tube or the like, on the outer circumferential face of the insertion tube. The rotation of the rotating tube creates a propulsive force by causing its fins to contact the inner wall of a lumen. With this propulsive force, the insertion tube moves in the insertion direction or the removal direction by itself. International Publication No. 2015/118773 also discloses that the endoscope system generates an index sound in accordance with the state associated with the propulsive force.

### BRIEF SUMMARY OF THE INVENTION

According to one aspect of the invention, the controller is a controller to control a first device, one of an endoscope and a treatment instrument being the first device. The other one of the endoscope and the treatment instrument is referred to as a second device. The endoscope includes an insertion tube and a self-propelled mechanism adapted to generate force for insertion or removal of the insertion tube. The controller includes a detection unit to detect a state of the second device, a determination unit to determine whether or not the second device is functioning based on an output signal of the detection unit, and a control unit to restrict an operation of the first device if the second device is determined to be functioning.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram showing an overview of a configuration example of a surgical system according to a certain embodiment.

FIG. 2 is a diagram showing an overview of a configuration example of a surgical system according to a first embodiment.

FIG. 3 is a diagram showing an example of noise of a high-frequency treatment instrument and encoder signals.

FIG. 4 is a flowchart showing an example of operations of a drive controller according to the first embodiment.

FIG. 5 is a flowchart showing an example of a rotation permissibility determination procedure according to the first embodiment.

FIG. 6 is a diagram showing an example of a PIP included in an image displayed by a display.

FIG. 7 is a diagram showing an overview of a configuration example of a surgical system according to a first modification of the first embodiment.

FIG. 8 is a flowchart showing an example of a rotation permissibility determination procedure according to the first modification of the first embodiment.

FIG. 9 is a flowchart showing another example of the rotation permissibility determination procedure according to the first modification of the first embodiment.

FIG. 10 is a diagram showing an overview of a configuration example of a surgical system according to a second modification of the first embodiment.

FIG. 11 is a diagram showing an overview of a configuration example of a surgical system according to a second embodiment.

FIG. 12 is a flowchart showing an example of a rotation permissibility determination procedure according to the second embodiment.

FIG. 13 is a flowchart showing an example of an output permissibility determination procedure according to the second embodiment.

FIG. 14 is a diagram showing an example of a PIP included in an image displayed by a display.

FIG. 15 is a diagram showing an overview of a configuration example of a surgical system according to a first modification of the second embodiment.

FIG. 16 is a flowchart showing an example of a rotation permissibility determination procedure according to the first modification of the second embodiment.

FIG. 17 is a diagram showing an overview of a configuration example of a surgical system according to a second modification of the second embodiment.

FIG. 18 is a diagram showing an overview of a configuration example of a surgical system according to a third embodiment.

FIG. 19 is a diagram showing an overview of a configuration example of a surgical system according to a modification of the third embodiment.

### DETAILED DESCRIPTION OF THE INVENTION

[Overview of Surgical System]

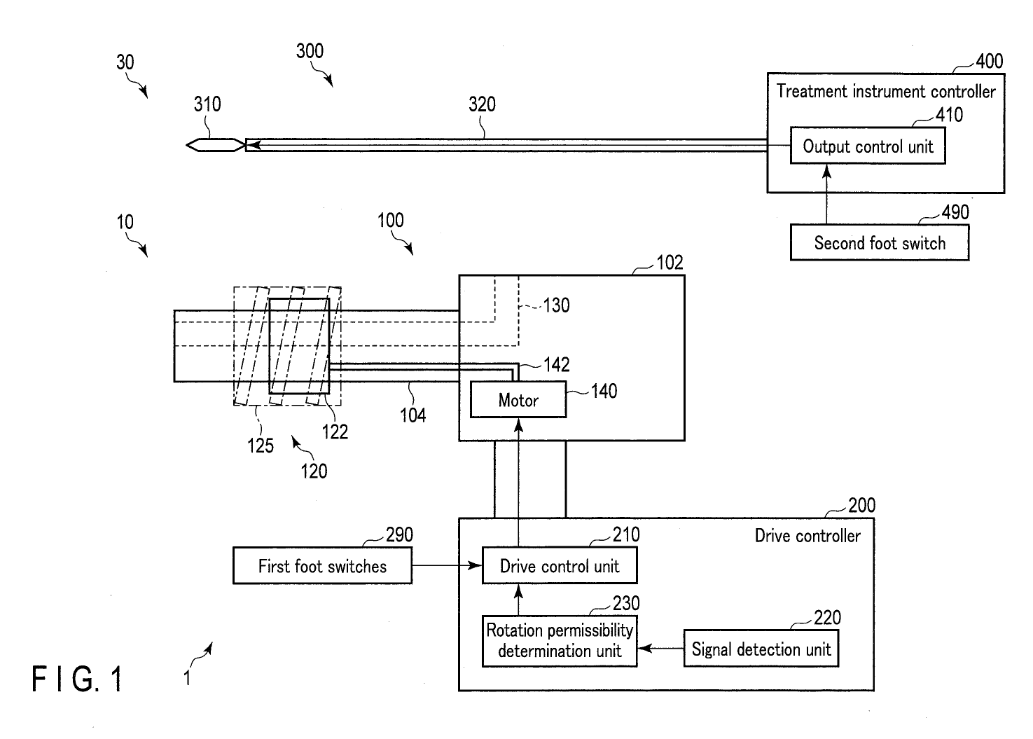

An overview of a certain embodiment of the present invention will be described with reference to FIG. 1. FIG. 1 shows a schematic configuration example of a surgical system 1. The surgical system 1 includes a self-propelled type endoscope system 10 for observation, and a treatment system 30 for treatment of affected sites.

The endoscope system 10 includes an endoscope 100. The endoscope 100 includes a control body 102 for a user to grasp for endoscope manipulations, and an insertion tube 104 to be inserted into, for example, the body of a patient. The insertion tube 104 is of an elongated shape and flexible. The insertion tube 104 is provided with a self-propelled mechanism 120. The self-propelled mechanism 120 includes a rotation unit 122. Around the rotation unit 122, a spiral tube 125 with helical fins is provided. The spiral tube 125 is removable from the rotation unit 122 and also, for example, is disposable.

The control body 102 is provided with a motor 140. The motor 140 generates a driving force for the self-propelled mechanism 120. Upon rotation of the motor 140, the rotary movement thereof is transmitted to the rotation unit 122 via a transmission member 142. The spiral tube 125 rotates according to the rotation of components in the rotation unit 122. In the state where the insertion tube 104 has been inserted into a lumen, etc., the helical fins of the spiral tube 125 catch and pull the tissue inside the lumen so that the insertion tube 104 is moved forward or backward. That is, the self-propelled mechanism 120 generates a force that enables the insertion or the removal of the insertion tube 104 of the endoscope 100.

The endoscope 100 further includes a instrument channel 130 that extends from the control body 102 to the distal end of the insertion tube 104. A tool such as forceps or a treatment instrument is inserted into the instrument channel 130 on the side of the control body 102, and comes out from the instrument channel 130 on the side of the distal end of the insertion tube 104. With the treatment instrument or the like protruding from the distal end of the insertion tube 104, a user can perform treatment operations at the site of the distal end of the insertion tube 104.

The endoscope system 10 also includes a drive controller 200 and first foot switches 290 for controlling operations of the self-propelled mechanism 120. The first foot switches 290 are switches for a user to provide inputs for the self-propelled mechanism 120 to operate.

Specifically, the first foot switches 290 include a forward pedal and a backward pedal. For example, the first foot switches 290 are configured so that the forward pedal is stepped on when a user desires to advance the insertion tube 104, that is, to move the insertion tube 104 in its distal end direction, and that the backward pedal is stepped on when a user desires to retract the insertion tube 104, that is, to move the insertion tube 104 in its proximal end direction.

While not illustrated in FIG. 1, the insertion tube 104 of the endoscope 100 is provided with an image sensor at its distal end. The endoscope 100 uses this image sensor to image the distal side from the insertion tube 104, and obtains image data of a subject. The acquired image data is processed by a video processor (not illustrated), and images of the subject are displayed by a display (not illustrated).

The drive controller 200 includes a drive control unit 210, a signal detection unit 220, and a rotation permissibility determination unit 230. The drive control unit 210 controls the rotation of the motor 140.

Specifically, the drive control unit 210 supplies current to the motor 140 according to the inputs to the first foot switches 290. For example, in response to detecting that the forward pedal has been stepped on, the drive control unit 210 supplies a current to the motor 140 for rotating the spiral tube 125 in the direction for moving the insertion tube 104 forward. Similarly, in response to detecting that the backward pedal has been stepped on, the drive control unit 210 supplies a current to the motor 140 for rotating the spiral tube 125 in the direction for moving the insertion tube 104 backward. The drive control unit 210 may determine a value of the current to be supplied to the motor 140 based on the amount of depression of the first foot switches 290.

The signal detection unit 220 acquires a signal associated with a state due to which the operations of the self-propelled mechanism 120 should be restricted. The signal detection unit 220 sends the acquired signal to the rotation permissibility determination unit 230. Examples of the state due to which the operations of the self-propelled mechanism 120 should be restricted include a state where the treatment system 30 is functioning, as will be described.

The rotation permissibility determination unit 230 determines whether or not the operations of the self-propelled mechanism 120 will be restricted, that is, whether or not the rotation of the rotation unit 122 should be permitted, based on the signal from the signal detection unit 220. The rotation permissibility determination unit 230 sends the result of determination to the drive control unit 210. The drive control unit 210 controls the rotation of the motor 140 based on the acquired determination result from the rotation permissibility determination unit 230. That is, if the rotation permissibility determination unit 230 has determined that the rotation of the rotation unit 122 should not be permitted, the drive control unit 210 does not rotate the motor 140 irrespective of the inputs to the first foot switches 290. On the other hand, if the rotation permissibility determination unit 230 has determined that the rotation of the rotation unit 122 should be permitted, the drive control unit 210 rotates the motor 140 according to the inputs to the first foot switches 290.

The treatment system 30 may be, for example, an electrosurgical knife adapted to output a high-frequency power, a laser treatment instrument, an argon plasma coagulator (APC), and so on. The treatment system 30 includes a treatment instrument 300, a treatment instrument controller 400, and a second foot switch 490.

The treatment instrument 300 includes, for example, an elongated insertable portion 320 adapted to run through the instrument channel 130 of the endoscope 100, and a distal end treatment portion 310 at the distal end of the insertable portion 320. If the treatment system 30 adopts an electrosurgical knife, the distal end treatment portion 310 is an electrode. If the treatment system 30 adopts a laser treatment instrument, the distal end treatment portion 310 is a laser probe. If the treatment system 30 adopts an APC, the distal end treatment portion 310 is an APC probe.

The second foot switch 490 is a switch for a user to provide inputs for the output of the treatment instrument 300 to be on and off. For example, the second foot switch 490 is configured so that it is stepped on when a user desires to output energy from the treatment instrument 300.

The treatment instrument controller 400 controls the output of the treatment instrument 300. The treatment instrument controller 400 includes an output control unit 410. The output control unit 410 controls supply of energy to the distal end treatment portion 310 according to the inputs to the second foot switch 490.

In the surgical system 1 as described, the operations of the self-propelled mechanism 120 of the endoscope 100 are restricted when, for example, the treatment instrument 300 is in operation for outputting the energy. As a result, the insertion tube 104 of the endoscope 100 is prevented from moving forward or backward during the operation of the treatment instrument 300. Therefore, it is possible to avoid the possibility of the user having to deal with the occurrence of unintentional movement of the treatment instrument 300 during its operation.

### First Embodiment

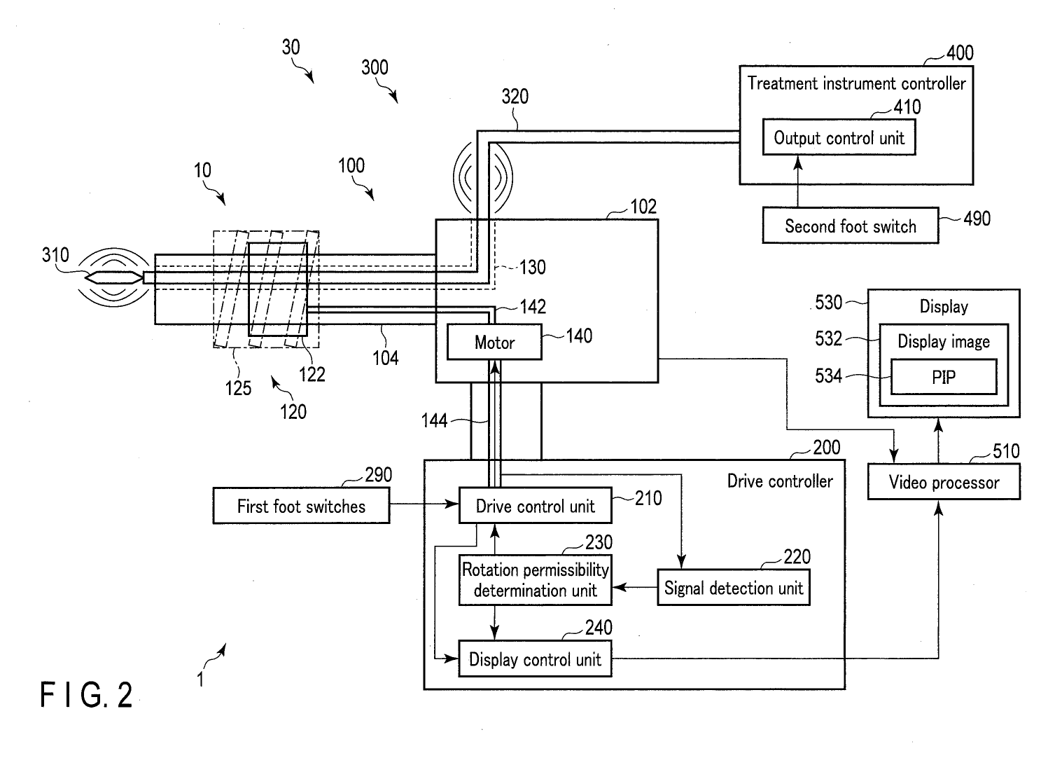

A first embodiment of the surgical system 1 will be described. The description will focus on the differences from the above overview. The same reference symbols will be used for the same components, etc., and their overlapping explanations will be omitted. FIG. 2 shows a schematic configuration example of the surgical system 1 according to this embodiment. In FIG. 2, the insertable portion 320 of the treatment instrument 300 has been inserted into the instrument channel 130 of the endoscope 100, and the distal end treatment portion 310 protrudes from the distal end of the endoscope 100.

In the example shown in FIG. 2, the surgical system 1 includes a video processor 510, and a display 530 as a display device. The video processor 510 acquires, from the endoscope 100, the image data that has been obtained by imaging at the endoscope 100. The video processor 510 performs image processing on the acquired image data to generate display image data. The video processor 510 causes the display 530 to display the image taken by the endoscope 100 as a display image 532, based on the display image data.

Also, in the example shown in FIG. 2, the drive controller 200 includes a display control unit 240. The display control unit 240 generates, as a display control signal, image data indicative of the information about the drive controller 200. This information may include, for example, a torque for the motor 140, the result of determination by the rotation permissibility determination unit 230, and so on. The display control unit 240 sends the image data for these information items to the video processor 510. Based on the image data acquired from the display control unit 240, the video processor 510 incorporates the information about the drive controller 200 into the display image 532 as a picture-in-picture (PIP).

The signal detection unit 220 according to this embodiment acquires signals which are attributable to the output of the treatment instrument 300 and which are superposed on a signal line 144 intended for the drive control unit 210 to control the motor 140. For example, if the treatment instrument 300 is a high-frequency treatment instrument such as an electrosurgical knife, a high-frequency current flows through the insertable portion 320 and the distal end treatment portion 310 of the treatment instrument 300. Noise attributed to this high-frequency current is superposed on the signal line 144 connecting the motor 140 and the drive control unit 210. The signal detection unit 220 detects this noise.

Assuming that the drive control unit 210 is adapted to control the operations of the motor 140 based on an output of an encoder (not illustrated) of the motor 140, the signal detection unit 220 acquires the output of this encoder in order to detect the noise attributed to the high-frequency current of the treatment instrument 300.

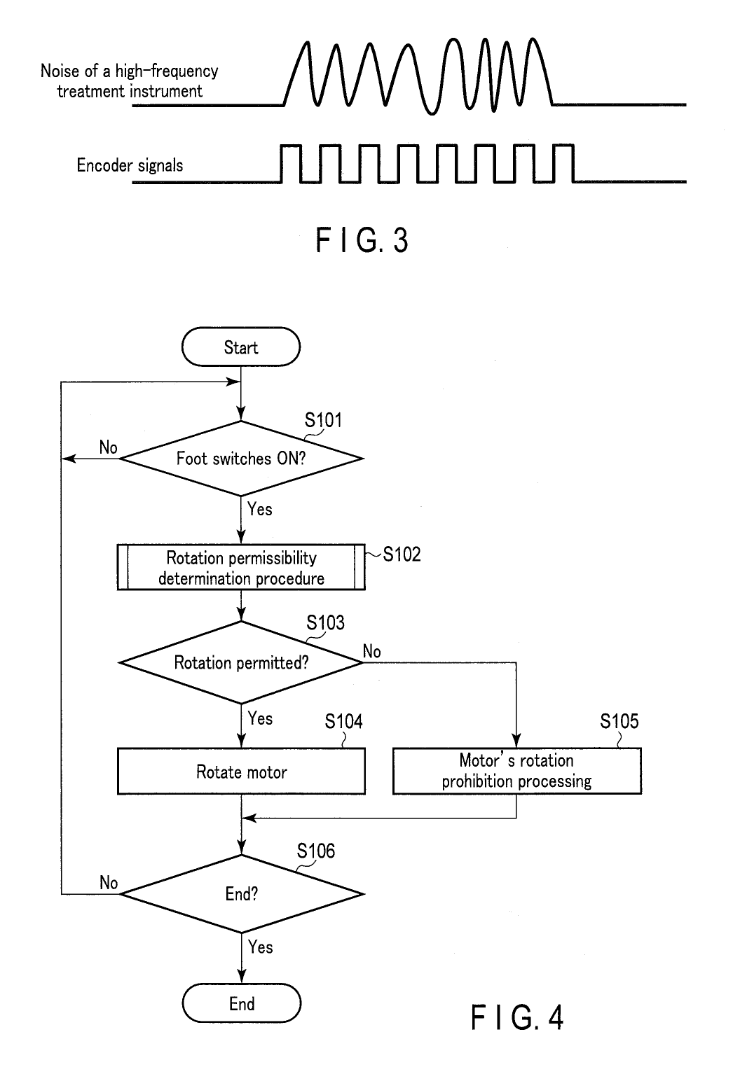

For example, it will be supposed that the encoder is influenced by the noise attributed to the high-frequency current of the treatment instrument 300, and this noise has the waveform as shown in the upper part of FIG. 3. In this instance, the encoder outputs pulses of a frequency corresponding to the frequency of the high-frequency current as shown in, for example, the lower part of FIG. 3. The signal detection unit 220 analyzes this output of the encoder and analyzes whether or not the output involves the noise due to the high-frequency current.

Examples of the drive control unit 210, the signal detection unit 220, the rotation permissibility determination unit 230, and the display control unit 240 in the drive controller 200 include an integrated circuit such as a central processing unit (CPU), an application specific integrated circuit (ASIC), or a field programmable gate array (FPGA). The drive control unit 210, the signal detection unit 220, the rotation permissibility determination unit 230, and the display control unit 240 may be each constituted by a single integrated circuit, etc., or by a combination of multiple integrated circuits, etc. Also, two or more of the drive control unit 210, the signal detection unit 220, the rotation permissibility determination unit 230, and the display control unit 240 may be constituted by a single integrated circuit, etc.

These integrated circuits are operated, for example, according to programs stored in a storage device in the drive controller 200 or the storage regions in the integrated circuits. The output control unit 410 in the treatment instrument controller 400 likewise includes an integrated circuit, etc.

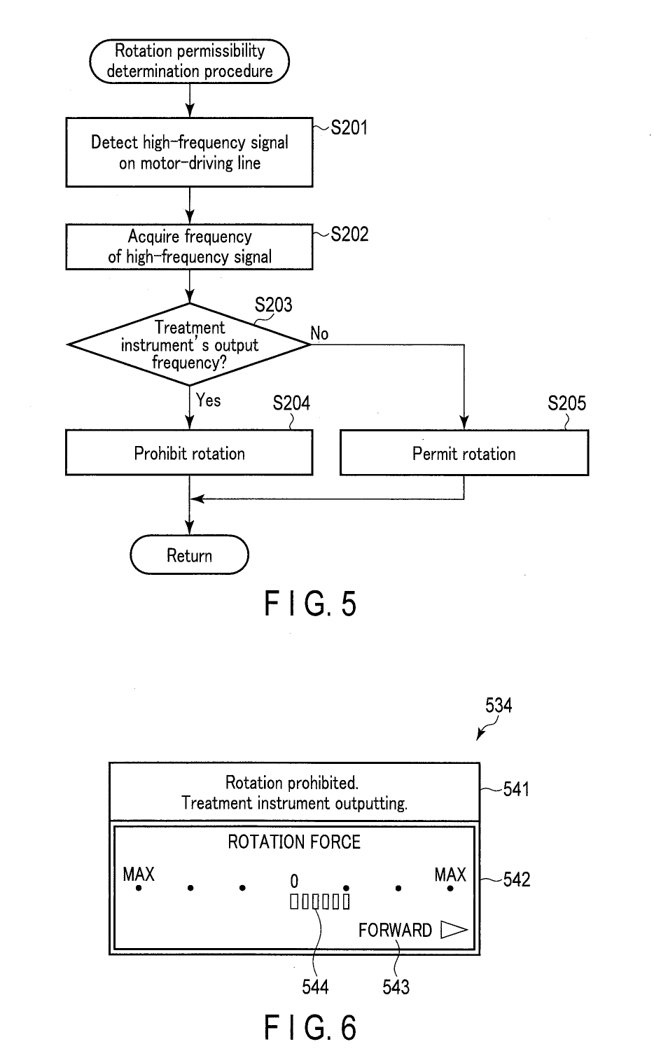

The operations of the drive controller 200 according to the present embodiment will be described with reference to the flowcharts given in FIGS. 4 and 5. The processing in FIG. 4 starts upon, for example, the drive controller 200 being powered on.

In step S101, the drive control unit 210 of the drive controller 200 determines whether or not the first foot switches 290 are turned on. When the first foot switches 290 are not on, step S101 is repeated so that the processing remains in a standby condition until the first foot switches 290 are on. Once the first foot switches 290 become on, the processing proceeds to step S102.

In step S102, the rotation permissibility determination unit 230 in the drive controller 200 performs a rotation permissibility determination procedure to determine whether or not the rotation unit 122 of the self-propelled mechanism 120 may be rotated. The rotation permissibility determination procedure is to determine whether or not the rotation of the rotation unit 122 should be permitted. The rotation permissibility determination procedure will be described later in more detail.

In step S103, the drive control unit 210 in the drive controller 200 determines whether or not the rotation has been permitted as a result of the rotation permissibility determination procedure. If the rotation has been permitted, the processing proceeds to step S104. In step S104, the drive control unit 210 in the drive controller 200 causes the motor 140 to rotate according to the input to the first foot switches 290. The processing then proceeds to step S106.

If it is determined in step S103 that the rotation has not been permitted, the processing proceeds to step S105. In step S105, motor rotation prohibition processing is performed for prohibiting the rotation of the motor 140. In this rotation prohibition processing, the drive control unit 210 may simply stop the supply of current to the motor 140 so that the motor 140 will not rotate. In another aspect of the rotation prohibition processing, the drive control unit 210 may also reduce the value of current supplied to the motor 140 so that the rotation of the motor 140 will slow down. Furthermore, in addition to regulating the motor 140 in such manners, a user may be informed that the operations of the self-propelled mechanism 120 are under restriction. By way of example, FIG. 6 will be referred to for the explanation of an instance where the display control unit 240 generates an image indicative of the operations of the self-propelled mechanism 120 subject to the restriction.

FIG. 6 shows an example of a PIP 534 included in the display image 532 displayed by the display 530. As shown in FIG. 6, the PIP 534 includes a warning display area 541 and a torque display area 542.

The torque display area 542 is displayed at all times throughout, for example, the operations of the endoscope system 10, and indicates the torque of the motor 140. The torque display area 542 includes a forward indication 543 to inform that the insertion tube 104 of the endoscope 100 is moving forward, and an indicator 544 to show the magnitude of the torque using a number of lights. As in the example shown in FIG. 6, when the insertion tube 104 is moving forward, the forward indication 543 arranged on the right side of the torque display area 542 lights up, and also one or more rectangles in the indicator 544 arranged on the right side of the torque display area 542 light up. If the insertion tube 104 is moving backward, a backward indication (not illustrated) arranged on the left side of the torque display area 542 lights up, and also one or more indicator rectangles arranged on the left side light up.

For example, when the output of the treatment instrument 300 is on, and the operations of the self-propelled mechanism 120 are prohibited, such information is given through the warning display area 541 as shown in FIG. 6. When no restrictions are imposed on the operations of the self-propelled mechanism 120, the warning display area 541 does not need to provide any information.

Turning back to FIG. 4, the description will continue. After the motor's rotation prohibition processing in step S105, the processing proceeds to step S106.

In step S106, the drive control unit 210 in the drive controller 200 determines whether or not to terminate the processing. For example, upon the drive controller 200 being powered off, the termination is determined. If the termination is not determined, the processing returns to step S101. If the termination is determined, the processing ends.

With reference to FIG. 5, the rotation permissibility determination procedure performed in step S102 will be described.

In step S201, the rotation permissibility determination unit 230 detects the high-frequency signal on a drive line for the motor 140, i.e., the signal line 144. In step S202, the rotation permissibility determination unit 230 acquires the frequency of the high-frequency signal.

In step S203, the rotation permissibility determination unit 230 determines whether or not the acquired frequency is an output frequency of the treatment instrument 300. If it is the output frequency, the processing proceeds to step S204. For example, assuming that the treatment instrument 300 is a high-frequency treatment instrument such as an electrosurgical knife, the acquired signal frequency may be determined to be the output frequency of the treatment instrument 300 if the signal frequency is greater than 10 kHz. In step S204, the rotation permissibility determination unit 230 determines that the rotation should not be permitted. The processing then returns to the main flow.

If it is determined in step S203 that the acquired frequency is not the output frequency, the processing proceeds to step S205. In step S205, the rotation permissibility determination unit 230 determines that the rotation should be permitted. The processing then returns to the main flow.

According to the present embodiment, whether or not the treatment instrument 300 is in operation for outputting is determined by detecting the high-frequency signal on the signal line 144 which is a drive line for the motor 140. If the treatment instrument 300 is in operation for outputting, the operations of the self-propelled mechanism 120 are restricted. Therefore, the embodiment can prevent the occurrence of the event where the insertion tube 104 of the endoscope 100—that is, the location of the distal end treatment portion 310 of the treatment instrument 300—is moved during the outputting operation of the treatment instrument 300 to induce an unintentional treatment behavior.

Note that the above descriptions of the embodiment have given the examples where the operative motions of the self-propelled mechanism 120 of the endoscope 100 are restricted according to the operations of the treatment instrument 300. In a similar fashion, the surgical system 1 may be configured so that the operative motions of the treatment instrument 300 are restricted according to the operations of the self-propelled mechanism 120. That is, the treatment instrument controller 400 may acquire the information about the operations of the self-propelled mechanism 120 of the endoscope 100, and restrict the operative motions of the treatment instrument 300 if the self-propelled mechanism 120 is in operation. With such a configuration, too, the embodiment can prevent the occurrence of the event where the treatment instrument 300 is allowed to output while the insertion tube 104 of the endoscope 100 is moving forward or backward.

For the embodiment, the self-propelled mechanism 120 has been exemplified using a configuration that the rotation of the spiral tube 125 causes the insertion tube 104 of the endoscope 100 to move forward or backward. However, the configuration of the self-propelled mechanism 120 is not limited to this. For example, the self-propelled mechanism 120 may adopt a configuration in which a belt is provided around the insertion tube 104 and the belt is rotated in the longitudinal direction of the insertion tube 104 for displacement toward the distal side or the proximal side, so that the insertion tube 104 is caused to move forward or backward.

The embodiment has been described using an example of displaying the state of the self-propelled mechanism 120 as a PIP on the display 530. This is not a limitation. For example, it is also suitable to provide a discrete display for exclusively displaying information equivalent to the information displayed as the aforementioned PIP, separately from the display 530 adapted to present the images acquired by the endoscope 100.

[First Modification of First Embodiment]

A first modification of the first embodiment will be described. The description will focus on the differences from the first embodiment. The same reference symbols will be used for the same components, etc., and their overlapping explanations will be omitted.

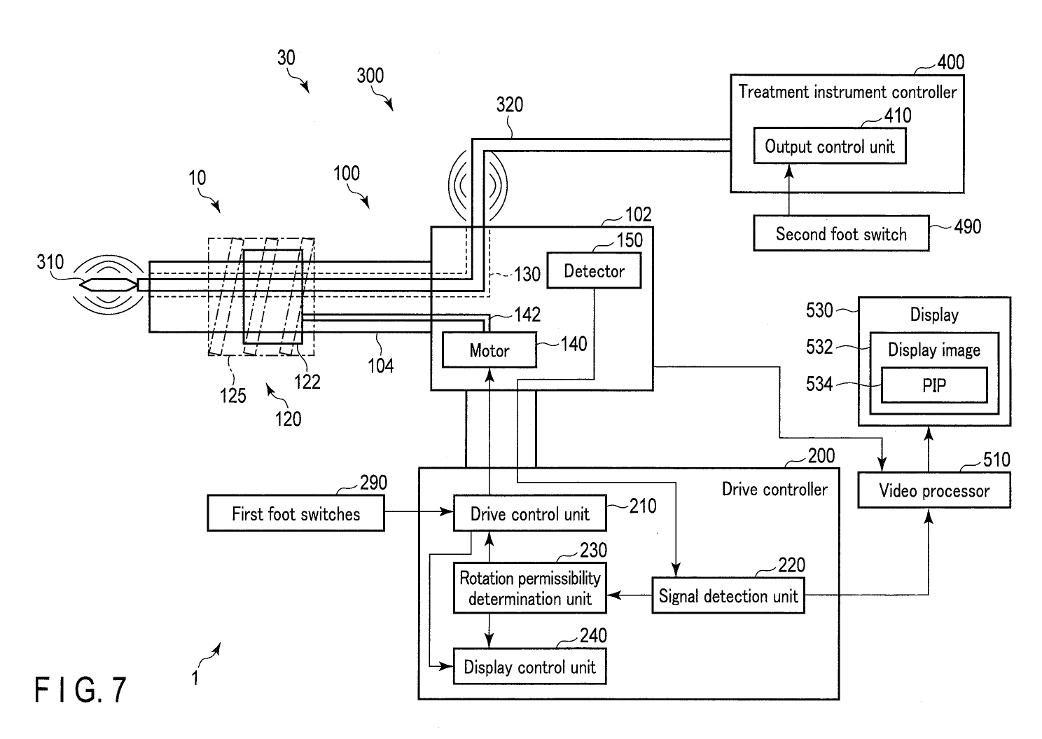

According to this modification as shown in FIG. 7, the endoscope 100 includes a detector 150 for detecting the presence or absence of the output of the treatment instrument 300. The detector 150 may be at any part of the endoscope. The detector 150 may be disposed at the control body 102 or at the insertion tube 104.

The detector 150 may include, for example, an antenna. For example, assuming that the treatment instrument 300 is a high-frequency treatment instrument, the treatment instrument 300 radiates electromagnetic waves according to the high-frequency current flowing through the treatment instrument 300. The detector 150 with the antenna detects the electromagnetic waves generated when the high-frequency treatment instrument 300 is in operation.

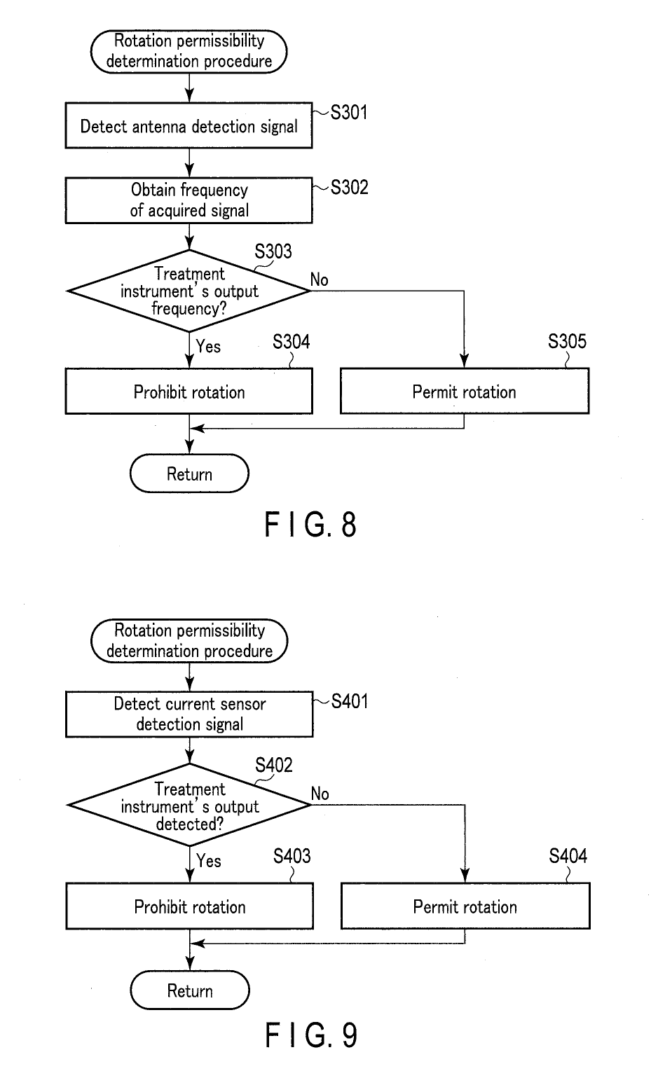

With reference to FIG. 8, a description will be given of the rotation permissibility determination procedure when the detector 150 adopts an antenna.

In step S301, the rotation permissibility determination unit 230 acquires, from the antenna detector 150, a signal that has been received through the antenna. In step S302, the rotation permissibility determination unit 230 subjects the acquired signal to the frequency analysis.

In step S303, the rotation permissibility determination unit 230 determines whether or not the acquired frequency is the output frequency of the treatment instrument 300. If it is the output frequency, the processing proceeds to step S304. In step S304, the rotation permissibility determination unit 230 determines that the rotation should not be permitted. The processing then returns to the main flow.

If it is determined in step S303 that the acquired frequency is not the output frequency, the processing proceeds to step S305. In step S305, the rotation permissibility determination unit 230 determines that the rotation should be permitted. The processing then returns to the main flow.

According to this modification, effects similar to those in the first embodiment can be obtained.

The detector 150 is not limited to an antenna, but may be a current sensor, a magnetic sensor, or the like. For example, when a current sensor is adopted, it may be disposed at any part of the output circuit ranging from the treatment instrument controller 400 to the distal end treatment portion 310. In the case of a magnetic sensor, for example, it may be disposed at a position in the output circuit ranging from the treatment instrument controller 400 to the distal end treatment portion 310, where generation of magnetism likely occurs when an electric current flows.

With reference to FIG. 9, a description will be given of the rotation permissibility determination procedure when the detector 150 adopts, for example, a current sensor.

In step S401, the rotation permissibility determination unit 230 acquires, from the current-sensor detector 150, a signal that has been detected by it. In step S402, the rotation permissibility determination unit 230 estimates whether or not the output of the treatment instrument 300 has been detected. If it is determined that the output of the treatment instrument 300 has been detected, the processing proceeds to step S403. In step S403, the rotation permissibility determination unit 230 determines that the rotation should not be permitted. The processing then returns to the main flow.

If it is determined in step S402 that the output of the treatment instrument 300 has not been detected, the processing proceeds to step S404. In step S404, the rotation permissibility determination unit 230 determines that the rotation should be permitted. The processing then returns to the main flow.

According to this modification, effects similar to those in the first embodiment can be obtained.

[Second Modification of First Embodiment]

A second modification of the first embodiment will be described. The description will focus on the differences from the first embodiment. The same reference symbols will be used for the same components, etc., and their overlapping explanations will be omitted.

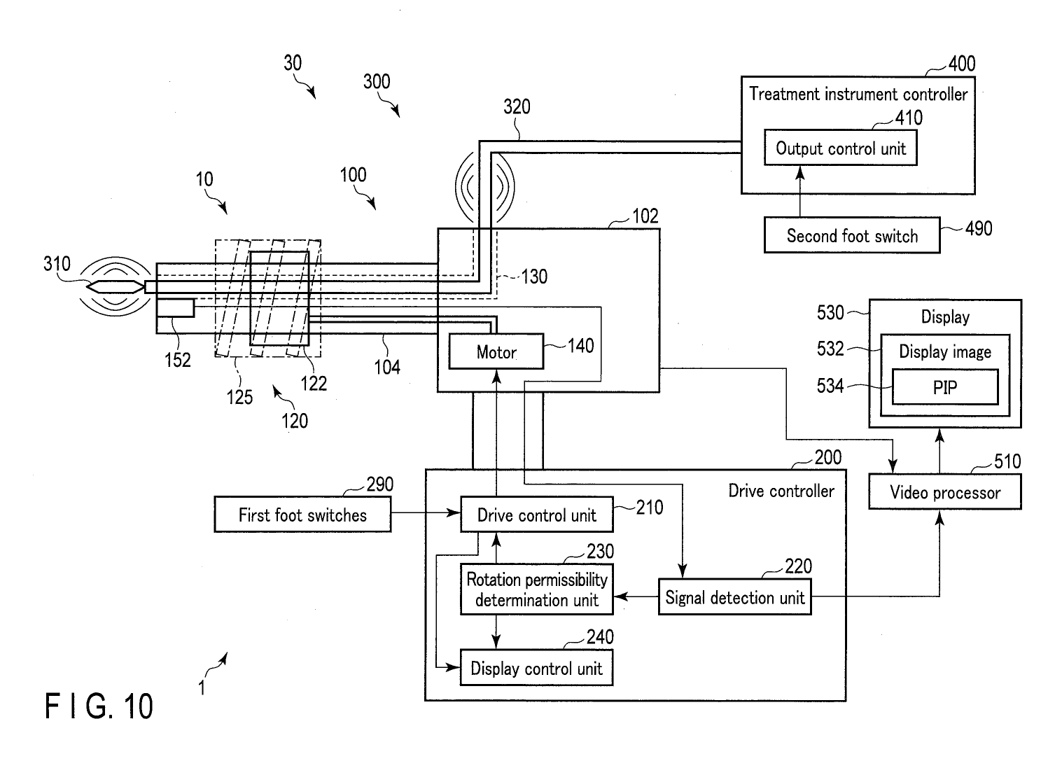

According to this modification as shown in FIG. 10, the endoscope 100 includes, at the instrument channel 130, an insertion sensor 152 for detecting the inserted state of the treatment instrument 300. From the output of the insertion sensor 152, the signal detection unit 220 acquires a signal indicative of whether or not the treatment instrument 300 has been inserted into the instrument channel 130, and sends information of the signal to the rotation permissibility determination unit 230.

The rotation permissibility determination unit 230 determines whether or not the self-propelled mechanism 120 should be permitted to make the rotating motions, based on the signal information acquired from the signal detection unit 220.

In the example shown in FIG. 10, the insertion sensor 152 is provided at the distal end portion of the insertion tube 104 of the endoscope 100. The insertion sensor 152 provided in this manner allows for the determination as to whether or not the treatment instrument 300 protrudes from the distal end of the insertion tube 104 of the endoscope 100. By referring to the result of this determination, it is possible to prevent the insertion tube 104 from being moved by the operation of the self-propelled mechanism 120 when the treatment instrument 300 protrudes from the distal end of the insertion tube 104.

According to this modification, furthermore, whether or not the treatment instrument 300 protrudes from the distal end of the insertion tube 104 can be determined using the insertion sensor 152 separately from the output of the treatment instrument 300, that is, regardless of the current flowing through the treatment instrument 300. Therefore, this modification of the embodiment is applicable to even the cases where the treatment instrument 300 is, for example, a pair of forceps or a knife which does not employ electrical mechanisms.

FIG. 10 shows the example where the insertion sensor 152 is provided at the distal end portion of the insertion tube 104, but this is not a limitation. The insertion sensor 152 may be arranged at the middle portion of the instrument channel 130. In this case, information as to whether or not the treatment instrument 300 protrudes from the distal end of the insertion tube 104 can be acquired by, for example, detecting how far the distal end of the treatment instrument 300 has been pushed toward the distal end side after passing by the position where the insertion sensor 152 is arranged.

### Second Embodiment

A second embodiment will be described. The description will focus on the differences from the first embodiment.

The same reference symbols will be used for the same components, etc., and their overlapping explanations will be omitted.

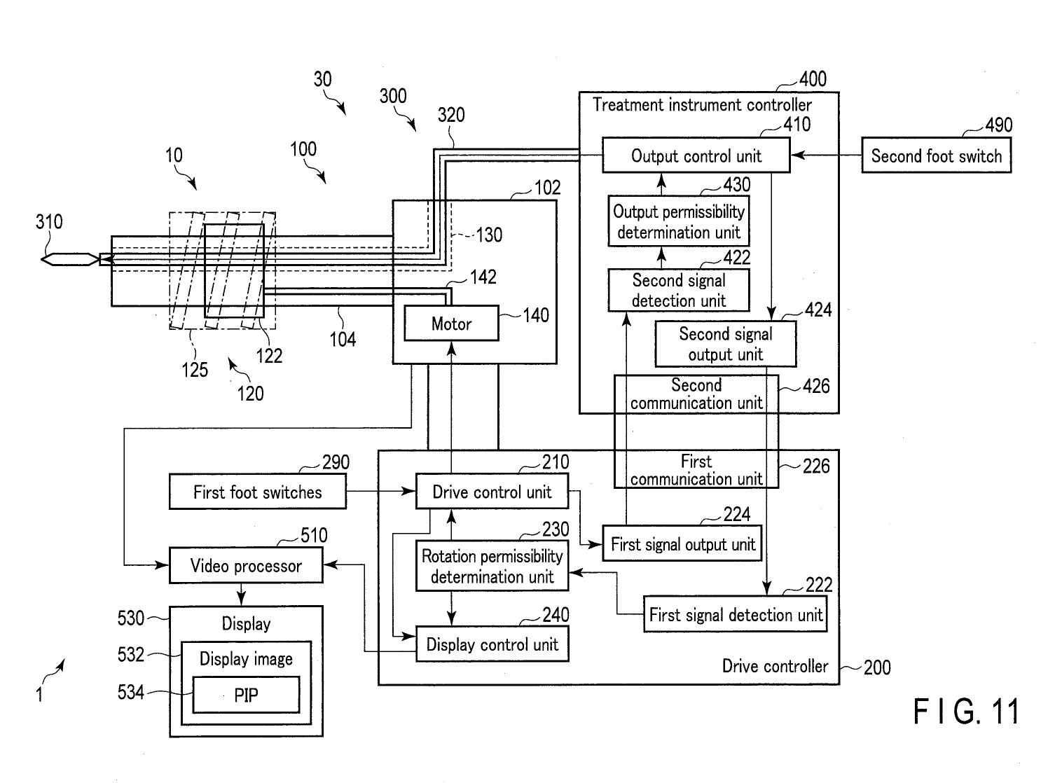

FIG. 11 shows a schematic configuration example of the surgical system according to the second embodiment.

According to this embodiment, the drive controller 200 for controlling the operations of the self-propelled mechanism 120 of the endoscope 100, and the treatment instrument controller 400 for controlling the output of the treatment instrument 300 are connected to each other for mutual communication and information exchange. Specifically, the drive controller 200 includes a first communication unit 226, and the treatment instrument controller 400 includes a second communication unit 426. The drive controller 200 and the treatment instrument controller 400 exchange information via the first communication unit 226 and the second communication unit 426.

The drive controller 200 includes a first signal detection unit 222 and a first signal output unit 224. The treatment instrument controller 400 includes a second signal detection unit 422 and a second signal output unit 424. The treatment instrument controller 400 further includes an output permissibility determination unit 430.

The second signal output unit 424 of the treatment instrument controller 400 outputs information about the output control unit 410 controlling the operations of the treatment instrument 300, to the drive controller 200. This information is acquired by the first signal detection unit 222 of the drive controller 200. The first signal detection unit 222 sends the acquired information to the rotation permissibility determination unit 230.

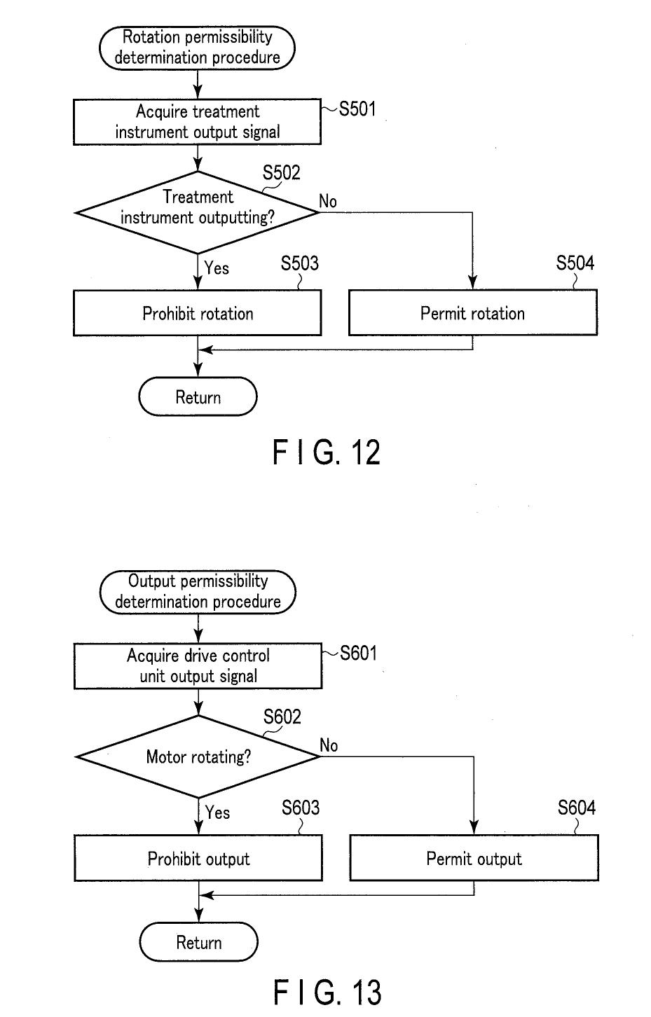

A description will be given of the rotation permissibility determination procedure performed by the rotation permissibility determination unit 230 according to this embodiment, with reference to the flowchart given as FIG. 12. In step S501, the rotation permissibility determination unit 230 acquires an output signal indicative of whether or not the treatment instrument 300 is in operation. In step S502, the rotation permissibility determination unit 230 estimates whether or not the treatment instrument 300 is in operation for outputting, based on the output signal. If it is determined that the outputting operation is ongoing, the processing proceeds to step S503. In step S503, the rotation permissibility determination unit 230 determines that the rotation of the rotation unit 122 should be prohibited, and terminates the rotation permissibility determination procedure. On the other hand, if it is determined in step S502 that the treatment instrument 300 is not in operation for outputting, the processing proceeds to step S504. In step S504, the rotation permissibility determination unit 230 determines that the rotation of the rotation unit 122 should be permitted, and terminates the rotation permissibility determination procedure.

The rotation permissibility determination unit 230 sends the result of determination as to whether or not to permit the rotation of the rotation unit 122 to the drive control unit 210. The drive control unit 210 controls the operation of the motor 140 based on the determination result. For example, if the rotation has been permitted, the drive control unit 210 causes the motor 140 to rotate in response to the first foot switches 290 stepped on and according to the amount of depression thereby occurred in the first foot switches 290. On the other hand, if the rotation has been prohibited, the drive control unit 210 does not cause the motor 140 to rotate, even when the first foot switches 290 are stepped on.

Turning to the drive controller 200, its first signal output unit 224 outputs information about the drive control unit 210 controlling the operations of the motor 140, to the treatment instrument controller 400. This information is acquired by the second signal detection unit 422 of the treatment instrument controller 400. The second signal detection unit 422 sends the acquired information to the output permissibility determination unit 430.

A description will be given of the output permissibility determination procedure by the output permissibility determination unit 430 according to this embodiment, with reference to the flowchart given as FIG. 13. In step S601, the output permissibility determination unit 430 acquires an output signal of the drive control unit 210 that is indicative of whether or not the motor 140 is in operation. In step S602, the output permissibility determination unit 430 estimates whether or not the motor 140 is rotating, based on the output signal. If it is determined that the motor 140 is rotating, the processing proceeds to step S603. In step S603, the output permissibility determination unit 430 determines that the outputting operation of the treatment instrument 300 should be prohibited, and terminates the output permissibility determination procedure. On the other hand, if it is determined in step S602 that the motor 140 is not rotating, the processing proceeds to step S604. In step S604, the output permissibility determination unit 430 determines that the output of the treatment instrument 300 should be permitted, and terminates the output permissibility determination procedure.

The output permissibility determination unit 430 sends the result of determination as to whether or not to permit the output of the treatment instrument 300 to the output control unit 410. The output control unit 410 controls the operation of the treatment instrument 300 based on the determination result. For example, if the output has been permitted, the output control unit 410 turns on the output of the treatment instrument 300 in response to the second foot switch 490 stepped on. On the other hand, if the output has been prohibited, the output control unit 410 does not turn on the output of the treatment instrument 300, even when the second foot switch 490 is stepped on.

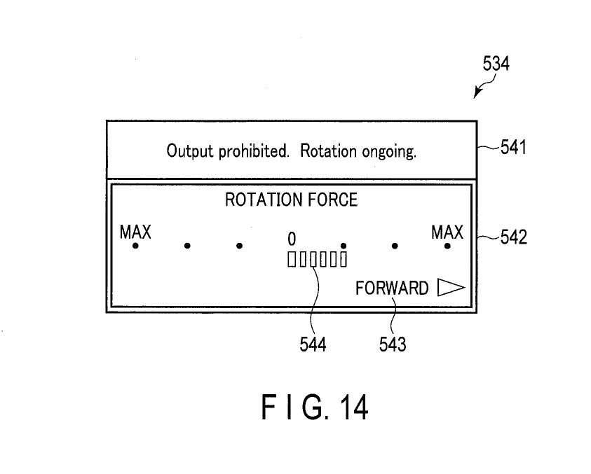

At this time, the display 530 displays the display image 532 containing the PIP 534. FIG. 14 shows one example of this PIP 534. The PIP 534 in FIG. 14 includes the warning display area 541 and the torque display area 542. For example, when the rotation unit 122 of the self-propelled mechanism 120 is rotating, and the output of the treatment instrument 300 is prohibited, such information is given through the warning display area 541 as shown in FIG. 14. Similarly to the earlier description, when the output of the treatment instrument 300 is on, and the operations of the self-propelled mechanism 120 are prohibited, such information may be given through the warning display area 541 as shown in FIG. 6. For example, when neither the first foot switches 290 nor the second foot switch 490 is stepped on, the warning display area 541 does not need to provide any information.

According to the present embodiment, if one of the self-propelled mechanism 120 and the treatment instrument 300 is already in operation, the other one is restricted from entering an operative state. As a result, the embodiment can prevent the occurrence of the event where the parallel operations of the self-propelled mechanism 120 and the treatment instrument 300 would cause the insertion tube 104 of the endoscope 100 to make a movement during the outputting operation of the treatment instrument 300 to consequently allow the treatment instrument 300 to influence unintended sites.

[First Modification of Second Embodiment]

A first modification of the second embodiment will be described. The description will focus on the differences from the second embodiment. The same reference symbols will be used for the same components, etc., and their overlapping explanations will be omitted.

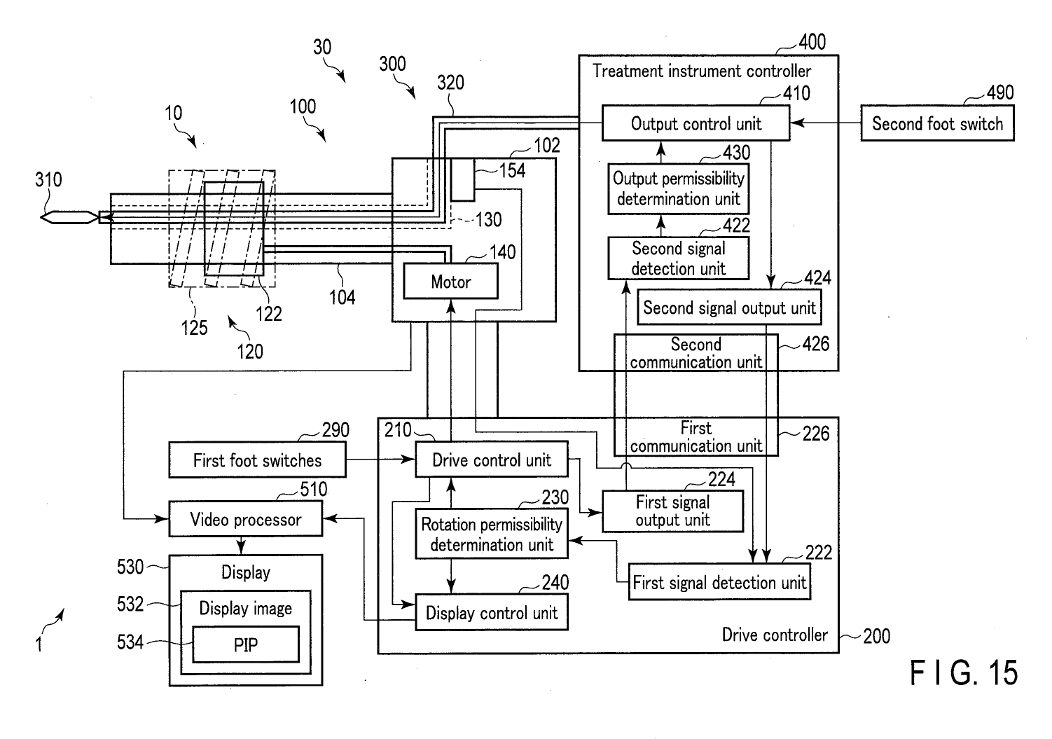

FIG. 15 shows a schematic configuration example of a surgical system according to the first modification of the second embodiment. According to this modification, an insertion sensor 154 is provided at the instrument channel 130. This insertion sensor 154 is adapted to detect whether or not the treatment instrument 300 has been inserted into the instrument channel 130. The insertion sensor 154 gives an output signal, which is sent to the rotation permissibility determination unit 230 via the first signal detection unit 222.

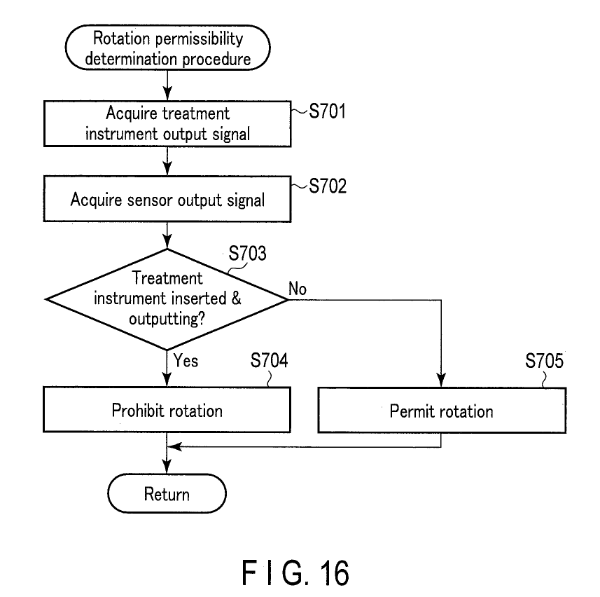

A description will be given of the rotation permissibility determination procedure performed by the rotation permissibility determination unit 230 according to this modification, with reference to the flowchart given in FIG. 16. In step S701, the rotation permissibility determination unit 230 acquires an output signal indicative of whether or not the treatment instrument 300 is in operation. In step S702, the rotation permissibility determination unit 230 also acquires the output signal of the insertion sensor 154. In step S703, the rotation permissibility determination unit 230 determines, based on the acquired signals, whether or not the treatment instrument 300 has been inserted into the instrument channel 130 and whether or not the treatment instrument 300 is in operation for outputting. If it is determined that the treatment instrument 300 has been inserted into the instrument channel 130 and also the treatment instrument 300 is in operation for outputting, the processing proceeds to step S704. In step S704, the rotation permissibility determination unit 230 determines that the rotation of the rotation unit 122 should be prohibited, and terminates the rotation permissibility determination procedure. On the other hand, if it is determined in step S703 that the treatment instrument 300 has not been inserted into the instrument channel 130, or that the treatment instrument 300 is not in operation for outputting, the processing proceeds to step S705. In step S705, the rotation permissibility determination unit 230 determines that the rotation of the rotation unit 122 should be permitted, and terminates the rotation permissibility determination procedure.

The rotation permissibility determination unit 230 sends the result of determination as to whether or not to permit the rotation of the rotation unit 122 to the drive control unit 210. The drive control unit 210 controls the operation of the motor 140 based on the determination result. For example, if the rotation has been permitted, the drive control unit 210 causes the motor 140 to rotate in response to the first foot switches 290 stepped on and according to the amount of depression thereby occurred in the first foot switches 290. On the other hand, if the rotation has been prohibited, the drive control unit 210 does not cause the motor 140 to rotate, even when the first foot switches 290 are stepped on.

According to the present modification, when, for example, the treatment instrument 300 is under an output test without being inserted into the instrument channel 130, the operations of the self-propelled mechanism 120 are not restricted even during the operations of the treatment instrument 300. However, when the treatment instrument 300 is operating while being inserted into the instrument channel 130, the operations of the self-propelled mechanism 120 are restricted.

[Second Modification of Second Embodiment]

A second modification of the second embodiment will be described. The description will focus on the differences from the second embodiment. The same reference symbols will be used for the same components, etc., and their overlapping explanations will be omitted. In the second embodiment, the first foot switches 290 are connected to the drive control unit 210, and the control signals of the drive control unit 210 are transmitted to the second signal detection unit 422 via the first signal output unit 224. Similarly, in the second embodiment, the second foot switch 490 is connected to the output control unit 410, and the control signals of the output control unit 410 are transmitted to the first signal detection unit 222 via the second signal output unit 424.

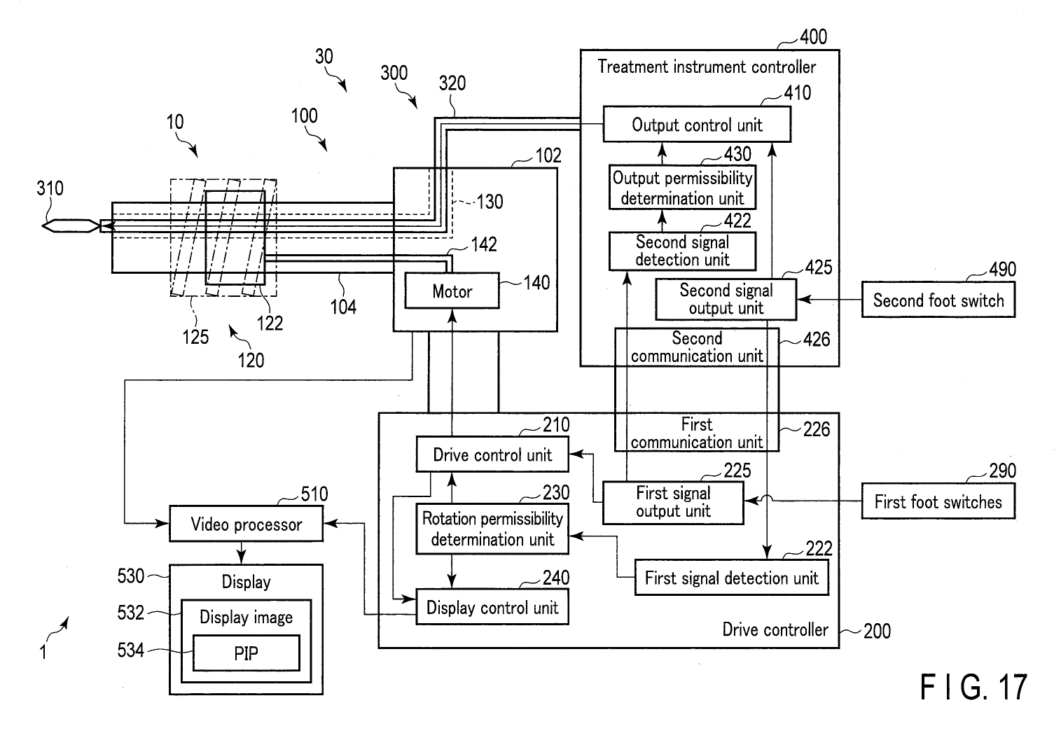

In contrast, this modification connects the first foot switches 290 to a first signal output unit 225 as shown in FIG. 17. The first signal output unit 225 sends the signal associated with the state of the first foot switches 290 to the drive control unit 210 as well as to the second signal detection unit 422. The drive control unit 210 controls the operations of the motor 140 according to the amount of depression occurred in the first foot switches 290, informed from the first signal output unit 225. The second signal detection unit 422 sends the information about the state of the first foot switches 290, acquired from the first signal output unit 225, to the output permissibility determination unit 430. The output permissibility determination unit 430 determines whether or not to permit the output of the treatment instrument 300 based on the state of the first foot switches 290, that is, according to the operation signals associated with the first foot switches 290, instead of the control signals of the drive control unit 210.

In this modification, similarly, the second foot switch 490 is connected to a second signal output unit 425. The second signal output unit 425 sends the signal associated with the state of the second foot switch 490 to the output control unit 410 as well as to the first signal detection unit 222. The output control unit 410 controls the operations of the treatment instrument 300 according to the amount of depression occurred in the second foot switch 490, informed from the second signal output unit 425. The first signal detection unit 222 sends the information about the state of the second foot switch 490, acquired from the second signal output unit 425, to the rotation permissibility determination unit 230. The rotation permissibility determination unit 230 determines whether or not to permit the rotation of the motor 140 based on the state of the second foot switch 490, that is, according to the operation signals associated with the second foot switch 490, instead of the control signals of the output control unit 410.

According to this modification, effects similar to those in the second embodiment can be obtained.

### Third Embodiment

A third embodiment will be described. The description will focus on the differences from the first embodiment. The same reference symbols will be used for the same components, etc., and their overlapping explanations will be omitted.

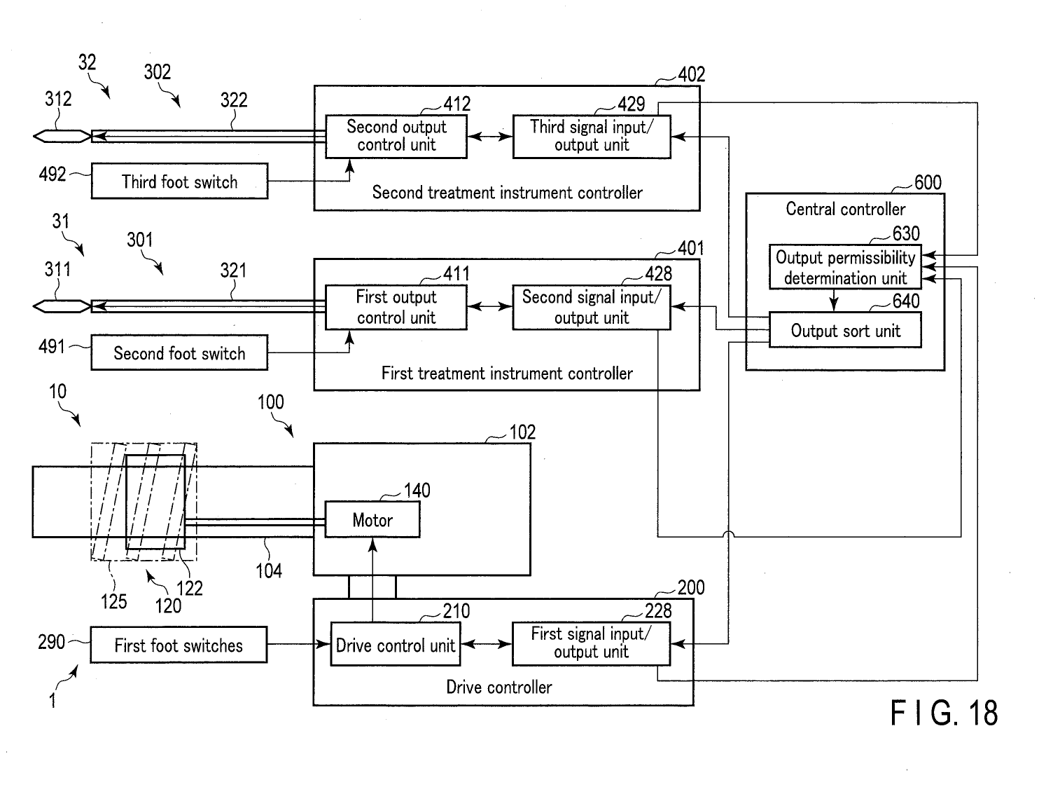

FIG. 18 shows a schematic configuration example of the surgical system 1 according to the third embodiment. The surgical system 1 according to this embodiment includes the endoscope system 10, a first treatment system 31, and a second treatment system 32. The first treatment system 31 and the second treatment system 32 are each similar to the treatment system 30 according to the first embodiment.

In the first treatment system 31, a first treatment instrument 301 includes a first insertable portion 321, and a first distal end treatment portion 311 at the distal end of the first insertable portion 321. Outputs of the first distal end treatment portion 311 are controlled by a first output control unit 411 of a first treatment instrument controller 401, according to inputs to a second foot switch 491. Similarly, in the second treatment system 32, a second treatment instrument 302 includes a second insertable portion 322, and a second distal end treatment portion 312 at the distal end of the second insertable portion 322. Outputs of the second distal end treatment portion 312 are controlled by a second output control unit 412 of a second treatment instrument controller 402, according to inputs to a third foot switch 492.

The surgical system 1 according to the present embodiment includes a central controller 600. The central controller 600 takes total control over the operations of the endoscope system 10, the first treatment system 31, and the second treatment system 32. The drive controller 200 in the endoscope system 10 includes a first signal input/output unit 228 so that it communicates with the central controller 600 via the first signal input/output unit 228. The first treatment instrument controller 401 in the first treatment system 31 includes a second signal input/output unit 428 so that it communicates with the central controller 600 via the second signal input/output unit 428. The second treatment instrument controller 402 in the second treatment system 32 includes a third signal input/output unit 429 so that it communicates with the central controller 600 via the third signal input/output unit 429.

The central controller 600 includes an output permissibility determination unit 630 and an output sort unit 640. The output permissibility determination unit 630 acquires control signal information from the drive control unit 210 via the first signal input/output unit 228. The output permissibility determination unit 630 acquires control signal information from the first output control unit 411 via the second signal input/output unit 428. The output permissibility determination unit 630 acquires control signal information from the second output control unit 412 via the third signal input/output unit 429. Based on the acquired information associated with the control signals of the drive control unit 210, the first output control unit 411, and the second output control unit 412, the output permissibility determination unit 630 determines the permissibility of the operations for each of the self-propelled mechanism 120, the first treatment instrument 301, and the second treatment instrument 302.

For example, if at least one of the first treatment instrument 301 and the second treatment instrument 302 is in operation, the output permissibility determination unit 630 determines that the operations of the self-propelled mechanism 120 should be prohibited. In another instance, if the self-propelled mechanism 120 is in operation, the output permissibility determination unit 630 determines that the operations of the first treatment instrument 301 and the second treatment instrument 302 should be prohibited.

The output permissibility determination unit 630 sends the determination results to the output sort unit 640. The output sort unit 640 outputs these determination results for output permissibility, to the drive controller 200, the first treatment instrument controller 401, and the second treatment instrument controller 402, respectively. The drive controller 200, the first treatment instrument controller 401, and the second treatment instrument controller 402 each perform individual output control based on the determination results for output permissibility acquired from the central controller 600.

According to the present embodiment, too, it is possible to prevent the occurrence of the event where the location of the first distal end treatment portion 311 or the second distal end treatment portion 312 is moved due to the operative motion of the self-propelled mechanism 120 during the operation of the first treatment instrument 301 or the second treatment instrument 302. Also, it is possible to prevent the occurrence of the event where the first treatment instrument 301 or the second treatment instrument 302 operates while the location of the first distal end treatment portion 311 or the second distal end treatment portion 312 is moving.

Note that the embodiment assumes use of two treatment systems. However, the embodiment is also applicable to cases where there is one treatment system as in the first embodiment, or there are three or more treatment systems.

[Modification of Third Embodiment]

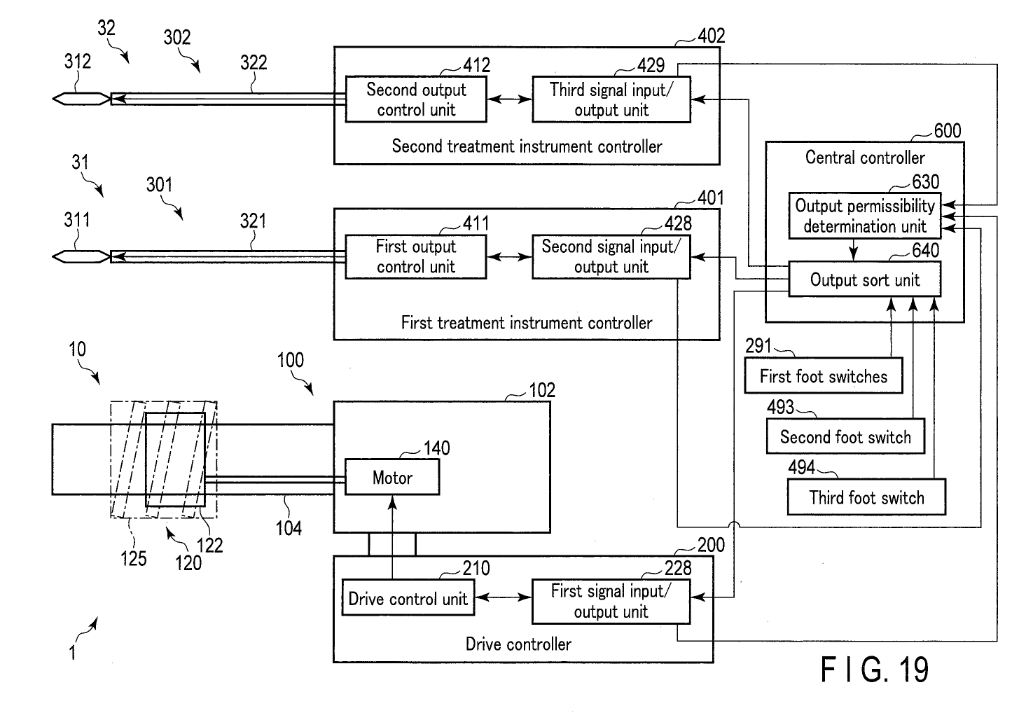

A modification of the third embodiment will be described. The description will focus on the differences from the third embodiment. The same reference symbols will be used for the same components, etc., and their overlapping explanations will be omitted. FIG. 19 shows a schematic configuration example of the surgical system 1 according to this modification. In this modification, first foot switches 291 for the inputs for operations of the self-propelled mechanism 120, a second foot switch 493 for the inputs for operations of the first treatment instrument 301, and a third foot switch 494 for the inputs for operations of the second treatment instrument 302 are connected to the output sort unit 640 of the central controller 600 so that the information about the states of these foot switches are acquired by the output sort unit 640. The output sort unit 640 sends the information about the states of these foot switches to the drive controller 200, the first treatment instrument controller 401, and the second treatment instrument controller 402, respectively. Other configurations are the same as those in the third embodiment.

According to this modification, effects similar to those in the third embodiment can be obtained.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A controller configured to control a first device, one of an endoscope and a treatment instrument being the first device, a remaining one of the endoscope and the treatment instrument being a second device, the endoscope comprising an insertion tube and a self-propelled mechanism adapted to generate force for insertion or removal of the insertion tube, the controller comprising:
at least one circuit configured to determine whether or not the second device is functioning; and
restrict an operation of the first device if the second device is determined to be functioning.

2. The controller according to claim 14, wherein
the first device is the endoscope,
the second device is the treatment instrument, the treatment instrument being a high-frequency treatment instrument adapted to output high-frequency power, and
the at least one circuit is configured to detect an output of the high-frequency treatment instrument,
determine that the second device is functioning if the output of the high-frequency treatment instrument is detected, and
control an operation of the self-propelled mechanism.

3. The controller according to claim 2, wherein the detector is configured to detect noise of the high-frequency power on a signal line for operating the self-propelled mechanism.

4. The controller according to claim 2, wherein
the endoscope further comprises an antenna adapted to detect an electromagnetic wave caused by the high-frequency power, and
the detector is configured to detect the output of the high-frequency treatment instrument based on a signal from the antenna.

5. The controller according to claim 14, wherein
the first device is the endoscope,
the second device is the treatment instrument, the treatment instrument being adapted to conduct a current,
the endoscope further comprises a current sensor adapted to detect the current or a magnetic sensor adapted to detect magnetism caused by the current,
the detector is configured to detect an output of the treatment instrument based on a signal from the current sensor or the magnetic sensor, and
the at least one circuit is configured to determine that the second device is functioning if the output of the treatment instrument is detected, and
control an operation of the self-propelled mechanism.

6. The controller according to claim 14, wherein
the first device is the endoscope,
the endoscope comprises a instrument channel,
the second device is the treatment instrument configured to be inserted into the instrument channel,
the endoscope further comprises an insertion sensor adapted to detect the treatment instrument inserted into the instrument channel,
the detector is configured to obtain an output of the insertion sensor, and
the at least one circuit is configured to determine that the treatment instrument is functioning if the treatment instrument protrudes from a distal end portion of the endoscope, and
control an operation of the self-propelled mechanism.

7. The controller according to claim 14, which is a first controller for controlling the first device, the second device being controlled by a second controller, wherein
the first controller is configured to communicate with the second controller,
the detector is configured to acquire the state of the second device from the second controller, and
the at least one circuit is configured to determine whether or not the second device is functioning based on the acquired state of the second device.

8. The controller according to claim 7, wherein the detector is configured to acquire an outputting state of the second device from the second controller.

9. The controller according to claim 7, wherein the detector is configured to acquire, from the second controller, an operation signal for operating the second device as a signal indicative of the state of the second device.

10. The controller according to claim 7, wherein
the first device is the endoscope,
the endoscope comprises a instrument channel,
the second device is the treatment instrument configured to be inserted into the instrument channel,
the endoscope further comprises an insertion sensor adapted to detect the treatment instrument inserted into the instrument channel,
the detector is further configured to detect an output of the insertion sensor, and
the at least one circuit is configured to determine that the treatment instrument is functioning if the treatment instrument protrudes from a distal end portion of the endoscope and if the acquired state of the second device indicates an operative state, and
control an operation of the self-propelled mechanism.

11. The controller according to claim 14, which is further configured to control an operation of the second device, wherein
the detector is configured to acquire a state of the first device and the state of the second device, and
the at least one circuit is configured to
determine whether or not each of the first device and the second device is functioning based on the acquired state of the first device and the acquired state of the second device, and
restrict the operation of the second device if the first device is determined to be functioning, and to restrict the operation of the first device if the second device is determined to be functioning.

12. The controller according to claim 1, wherein the at least one circuit is further configured to generate and output a display control signal, and
the at least one circuit generates and outputs a display control signal for a display to display information indicative of the operation of the first device being under restriction if the operation of the first device is restricted.

13. The controller according to claim 1, wherein the self-propelled mechanism comprises a helical fin around the insertion tube and along a longitudinal axis, a rotation unit configured to rotate the fin about the longitudinal axis, and a motor configured to rotate the rotation unit.

14. The controller according to claim 1, further comprising a detector configured to detect a state of the second device, wherein
the at least one circuit is configured to determine whether or not the second device is functioning based on an output signal of the detector.
